(19) United States
(12) Patent Application Publication
(10) Pub. No.: US 2004/0081577 A1
(43) Pub. Date: Apr. 29, 2004

(54) Method of treating comestible material for disinfestation, enzyme denaturation and microorganism control

(76) Inventors: Virgil J. Macaluso, Independence, KS (US); Joel J. Phillippi, Edina, MN (US); Russell W. Johanson, Ramsey, MN (US)

Correspondence Address: HOVEY WILLIAMS LLP, Suite 400, 2405 Grand, Kansas City, MO, 64108, US

(21) Appl. No.: 10/282,631

(22) Filed: Oct. 29, 2002

(51) Int. Cl. A61L 9/00; A61L 2/00

(52) U.S. Cl. 422/22; 422/1; 422/4; 422/5

(57) ABSTRACT

A method is provided for rapidly drying and treating comestible material. The method generally comprises heating the comestible material by application of infrared energy in order to dry the comestible material to a predetermined moisture content, and/or disinfest and denature enzymes within the comestible material. The heating step is carried out so that the maximum temperature of the comestible material does not exceed a predetermined level.

### BACKGROUND OF THE INVENTION

1. Field of the Invention

The present invention is broadly concerned with a method for rapidly drying rice and other comestible material. More particularly, the invention relates to a method whereby infrared energy is applied to the rice or comestible material thereby heating the rice or comestible material to no more than a predetermined temperature and drying the rice or comestible material to a predetermined moisture content within a relatively short time from the first application of infrared energy.

In addition to exhibiting excellent drying capabilities, the present invention is suitable for disinfesting the comestible material, inactivating microorganisms and/or fungi on the comestible material, and denaturing enzymes present within the comestible material.

2. Description of the Prior Art

In order to be stored for extended periods of time, rice and comestible material must be dried to a moisture content much less than that present at harvesting. Typically, freshly harvested or rough/paddy rice will have a moisture content of approximately 15-25% by weight. This moisture content level must be reduced prior to storing the rice otherwise the rice will mold or ferment. In order for the rice to be storage stable, it must have a moisture content no higher than 13% by weight and more preferably between about 10-12%. For other forms of comestible material specifically herbs such as tarragon and rosemary, spices, fruits such as blueberries, cranberries and kiwi, and vegetables, the moisture content immediately following harvest may be has high as 90% by weight rendering the material unsuitable for extended storage. More specifically, the moisture content of rosemary immediately following harvest will be about 58% by weight. Again, unless the moisture content level is reduced, the rosemary will degrade upon storage for extended periods of time. In order to be storage stable, the rosemary must be dried to a moisture content of less than about 10% by weight and more preferably between about 8-9%.

Currently, the conventional process for drying rice involves placing the rice in column driers using ventilation ports at various locations in the column wall, blowing hot air across the rice. The rice moves vertically through the column beginning at the top and is drawn from the bottom of the column by an auger, with the rice becoming drier as it approaches the bottom. However, it has been discovered that the rice nearer the column wall will be heated to a higher temperature than the rice nearer the column center. For this reason, rice cannot be dried continuously, instead, the rice must be allowed to intermittently cool down, rest or temper before further drying. It is important that the temperature of the rice not exceed 110° F. during this type of process or else the escaping moisture will induce stress in the rice kernel causing it to fracture, thus making the rice of a lower grade or quality. Typically, it takes about 3-5 days to dry rice according to this process.

The most widely used process for drying other comestible material consists of blowing warm air over the material on a multistage conveyor. For those other materials, care must be exercised during the drying process so that the materials are not damaged. For example, rosemary is typically dried in this fashion and while the temperature of the rosemary during the drying process is not as critical as that of rice, care must be exercised in heating the rosemary so as not to scorch it or cause degradation. The conventional process for drying rosemary is also time consuming as it can take up to 24 hours to dry rosemary to the required moisture content.

The use of nutriceuticals (dietary supplements, e.g. vitamins) has become increasingly popular as consumers have recognized health benefits from using these products. As used herein, “nutriceutical” refers to any nutritional supplement designed for any specific clinical purpose(s). Due to US regulations, clinical or medical claims cannot be made for them. Thus, presently all are functionally (legally) on the market as foods for general consumption (or “health foods”) to be used as “supplements” to nutrition (diet). Nutriceuticals are typically extracted from plants or portions thereof, for example, marigold leaves, grape skins, and grape seeds. Before the nutriceuticals could be extracted, the plant material must be dried. Conventionally, drying is accomplished by freeze drying the plant material. Freeze drying is an expensive process in which the material is subjected to temperatures between about −40-50° F. for long periods of time. Consequently, there is a need for a faster, less expensive method of drying the plant material from which the nutriceuticals are derived without damaging the nutrients themselves.

The infestation of comestible material with insects, microorganisms, and fungi is a continuing problem for the agriculture industry. Conventional methods for disinfesting comestible material have involved the use of pesticides for chemically treating the material. The most effective chemical used on stored grain has been methyl bromide, however this chemical has been banned by the U.S. Environmental Protection Agency.

Alternative, chemical-free methods for disinfesting comestible material include both low temperature and high temperature treatment. Exposing pests to both temperature extremes results in reduced insect reproduction and survival rates. However with low temperature treatment, some insects may become acclimatized and their cold-hardiness can increase by 2 to 10 times. Exposing pests to high temperatures 60°-65° C. (140°-149° F.) for short periods of time has proven effective in killing insects, however such temperature can damage the quality of the comestible material, therefore, material temperature must be carefully measured and controlled. Typically, high temperature treatment is accomplished by passing hot air over the material, however, material temperature is difficult to control with this technique.

Infrared technology has been used in the past in order to sterilize surfaces prone to bacterial and microbial growth. More specifically, this technology has become adapted for use in sterilizing the floors of food processing plants. However, the use of infrared technology in treating comestible material, until now, has not been a practical or commercially viable alternative. Therefore, there is a need in the art for an effective method of treating comestible material to inactivate microorganisms and fungi on the material without the use of chemicals and under conditions which will not harm the material quality.

Many countries, especially a number of European countries, have begun to require sterilization of animal feed, particularly in effort to control the spread of animal borne diseases such as mad cow disease. As more countries adopt similar requirements, a need has arisen for a fast, efficient method of animal food sterilization which will kill the disease causing microorganisms, but will not significantly affect the palatability or nutritional value of the animal feed.

Another problem with long-term storage of comestible material is the presence of certain enzymes which tend to cause rancidity of the material when stored for extended periods of time. One such enzyme is the enzyme lipase found, for example, in rice. Lipase is a strong enzyme in the rice pericarp which, when released into the bran during the milling process, causes rapid rancidity by formation of free fatty acids. Spoilage of comestible material caused by oxidative rancidity involves a reaction between the lipid and molecular oxygen. The reaction takes place at the double bonds of unsaturated fatty acids and can be accelerated by the action of other enzymes which contain a transition metal prosthetic group such as lipoxygenase (LOX). LOX is found in a variety of plants, particularly legumes, such as soybeans, mungbeans, navy beans, green beans, peas, and peanuts, and cereal, such as rye, wheat oat, barley, and corn.

In order to process comestible material, particularly cereal bran, into a high quality food grade product exhibiting good shelf-life, all components causing deterioration must be removed or their activity arrested. Therefore, it is very important that the inactivation of lipase and LOX be complete and irreversible. Conventionally, the bran would be extruded in order to stabilize it thereby improving the shelf life of material. This extra processing step adds additional expense to the overall cost. Therefore, it would be highly desirable to deactivate these enzymes before they can cause rancidity in the comestible material.

Some comestible materials, especially cereal grains must be aged prior to being incorporated into useful food products. Food manufacturers will often age rice, for example, for periods of between 60-90 days prior to using the rice in making finished food products. It has been discovered that this aging period leads to higher quality finished products. It is believed that the aging process transforms amylose and amylase components within the rice resulting in the improved properties of the rice. However, long aging times can significantly delay handling and processing of the comestible material leading to higher costs. Therefore, there is a real and unfulfilled need for a method of accelerating the aging process of comestible material.

### SUMMARY OF THE INVENTION

The present invention overcomes the problems outlined above and provides a fast, efficient method for drying rice, preferably rough/paddy rice, and other comestible material. Generally, the invention comprises heating the rice or other comestible material by application of infrared energy thereto in order to dry the rice or comestible material to a predetermined moisture content. The heating step is carried out for a maximum period of about six hours, and more preferably about three hours, so that the maximum temperature of the rice or other comestible material does not exceed a predetermined level.

As noted above, conventional column driers will produce a substantial temperature gradient within the material being dried. Material located closer to the hot air inlet side will generally have a higher temperature than material located closer to the air outlet side. The method of the present invention provides a reduced temperature gradient within the material, which results in a more uniform moisture content through the material being dried. The method of the present invention results in a temperature gradient present within the material being dried at any given point in the drying process is less than about 10%. Preferably this temperature gradient is less than about 5%, and more preferably less than about 3%.

When rice is being dried, it is preferable to heat the rice to a temperature of not more than about 110° F. and to dry it to a moisture content of between about 7-13% by weight. It is also preferable that the drying step results in a milling yield of no less than about 55/70 or 78%, more preferably about 60/70 or 86%, and most preferably about 64/70 or 91%. In order to determine the milling yield, the a sample of the dried rice is taken and the rice hull and bran are milled off leaving just the rice kernel. Each rice kernel is then individually examined and the fractured kernels removed. The milling yield is simply the weight ratio of unfractured rice kernels/total rice kernels. The same methodology may be applied to par-boiled rice in addition to freshly harvested or rough/paddy rice. Par-boiled rice, very generally, is rice that has undergone some degree of cooking. Par-boiled rice is produced by taking rice that may or may not have been dried previously and partially cooking it. The partially-cooked or par-boiled rice will have a moisture content of about 35% by weight which will need to be reduced to between about 7-13% if the par-boiled rice is to be stored while awaiting further processing. When par-boiled rice is being dried, it is preferable to heat the par-boiled rice to a temperature of not more than about 140° F. and even more preferably not more than about 110° F.

Preferred comestible material for use with the method of the present invention comprises herbs, spices, fruits, and vegetables. More preferably the comestible material comprises rosemary, garlic, onion, and capsicum. When most comestible material is being dried, temperature is not as critical a factor as it is for rice, however, even in these instances it is preferable to heat the comestible material to a temperature of not more than about 180° F. Depending upon desired characteristics of the dried material, the material could be dried to any predetermined moisture content. In most cases, the comestible material should be dried to a moisture content of between about 6-10% by weight and preferably to between about 8-9%.

The method of the present invention may be employed in either a continuous or a batch process and is particularly suited for use with an agitator-type drying apparatus which broadly includes an elongated housing presenting a material inlet, and a spaced material outlet, together with structure for exit of moisture from the housing. Preferably, an elongated, essentially imperforate, flexible bed is located within the housing and extends between the inlet and the outlet. In some forms, the bed presents an upper material supporting surface, an opposed lower surface, and a pair of side margins. The overall bed assembly further has an upstanding skirt member adjacent each of the side margins, with the bed and skirt members cooperatively retaining the material during heating and drying thereof. A series of agitators are located below and spaced along the length of the bed in order to generate an undulating bed movement serving to elevate and tumble the material on the bed and to move the material along the length thereof from the inlet to the outlet. Finally, a number of infrared heating units are disposed above the bed within the housing for heating and drying of the material as it is elevated and tumbled.

In one form of the agitator-type drying apparatus, the bed is mounted within the housing substantially in tension and against translational movement. In this embodiment, one end of the bed is preferably fixedly secured to the housing, whereas the other end is yieldably attached through the use of a spring or the like. Alternately, the bed may be in the form of an endless, essentially imperforate belt trained about endmost rollers.

The agitators are advantageously provided along substantially the entire length of the bed and are in the form of respective, rotatable, multiple-roller beater bar assemblies. A variable speed drive is advantageously coupled with the beater bar assemblies for rotation thereof at different rotational speeds at the discretion of the operator.

The skirt members forming a part of the bed can be constructed in a number of ways so long as the material-retaining function thereof is preserved. In one embodiment, the marginal ends of the flexible bed are turned upwardly and secured to connectors affixed to the housing sidewalls. Alternately, upstanding, elongated lengths of cloth material (preferably sail cloth) are affixed to the bed margins, with the upper ends of the cloth material lengths being supported by connectors secured to the housing walls. In the case of a continuous belt bed, the cloth skirt members are supported above the upper run of the belt and lie on the upper run.

In order to provide desirable operational flexibility, the housing is preferably provided with means for adjusting the inclination of the bed. Generally, a degree of inclination of plus or minus 6° from horizontal is sufficient, and this can be readily accommodated by a conventional jacking screw arrangement. Also, positive pressure ambient air can be introduced into the housing to assist in and control the drying of the rice or comestible material.

The present invention further provides for treating comestible material with infrared radiation, preferably far-infrared radiation, and more preferably infrared radiation having a wavelength of about 2-7 microns, in order to disinfest the material, inactivate fungi and microorganisms, and also denature enzymes within the material. As used herein with respect to fungi and microorganisms, the term “inactivate” means to kill, destroy, or render biologically inert. In order to achieve these objectives, comestible material is exposed to infrared radiation for short periods of time (preferably less than about 3 minutes, and more preferably less than about 30 seconds) while heating the material to a temperature up to about 300° F., and preferably between about 100°-200° F., and more preferably between about 140°-170° F. It is also desirable during the treatment process to expose substantially all of the outer surface of the comestible material to the infrared radiation. In order for the treatment to be fully effective, substantially all surfaces and the pests and microorganisms on those surfaces be exposed to the infrared radiation. This exposing step is preferably carried out in the same manner as describe above with respect to drying of comestible material. The material is preferably placed on an elongated, essentially imperforate flexible bed and exposed to the infrared radiation and heated. The lower surface of the bed is repeatedly impacted in order to generate an undulating bed movement serving to elevate and tumble the material on the bed and to move the material along the length thereof.

Comestible material may be treated with infrared radiation for disinfestation, inactivation of microorganisms and fungi, and denaturing of enzymes while undergoing the drying process as described above as long as the time and temperature requirements set forth for such treatment are satisfied. It is also possible to treat comestible material which has previously been dried or refined by exposing the material to infrared radiation for a sufficient time to disinfest the comestible material, inactivate microorganisms or fungi on the material, or denature enzymes within the material. The precise time and temperature requirements for treatment vary somewhat depending upon the material being treated and the treatment objective. For example, in treating comestible material in order to inactivate microorganisms, the particular temperature selection will depend at least in part on the particular microorganism targeted for destruction. Treatment of the material should be for limited duration to avoid damaging the material, preferably for less than about 3 minutes, and more preferably less than about 30 seconds. Generally, the higher the temperature at which the material is treated, the shorter the treatment time of the material. Such short treatment times are adequate to protect the overall quality of the material even if the temperature of the material rises beyond the maximum which may be reached during the drying process.

Any comestible material may be treated according to the invention, however preferred materials include those which are typically stored for extended periods of time, for example, grains such as rice, wheat, and barley, and refined products such a flour, granulated sugars, and corn meal.

The infrared radiation acts to kill insects, their eggs and larvae, bacteria and other microorganisms such as Escherichia coli, Staphylococcus aureus, Coleoptera sp. and Rhyzopertha dominica. Exposing the material to infrared radiation for sufficient time at a sufficiently high temperature will preferably inactivate at least about 95% of all pests, microorganisms, and fungi in the material, and more preferably inactivate at least about 99%, and most preferably about 100%.

Advantageously the infrared radiation denatures enzymes in the comestible material. For example, infrared radiation denatures lipase found in cereal bran, particularly rice bran. Lipase is a strong enzyme in the rice pericarp which, when released into the bran during the milling process, causes rapid rancidity by formation of free fatty acids. By denaturing these enzymes, comestible material shelf life, and particularly that of cereal bran, is improved.

The present invention is also useful in accelerating the aging process of comestible material, preferably cereal grains, and most preferably rice. By exposing the comestible material to far-infrared radiation and heating the material to temperatures of up to about 200° F., and more preferably between about 140-170° F., certain chemical components within the comestible material, namely amylose and amylase are transformed. This transformation imparts improved properties to the comestible materials which lead to higher quality food products. For example, in rice, transformation of amylose and amylase leads to improved cooking quality resulting in a less sticky rice product.

### BRIEF DESCRIPTION OF THE DRAWINGS

FIG. 1 is a side view in partial vertical section illustrating the construction of a preferred agitated bed infrared drying apparatus suited for use in accordance with the invention;

FIG. 2 is a sectional view taken along lines 2-2 of FIG. 1 and further illustrating the construction of the preferred drying apparatus.

FIG. 3 is a vertical sectional view taken along lines 3-3 of FIG. 1 and depicting the operation of the drying apparatus at a region thereof where a bed agitator is in its lower position;

FIG. 4 is a vertical sectional view taken along the lines 4-4 of FIG. 1 and depicting the operation of the drying apparatus at a region thereof where a bed agitator is in its upper position to elevate and tumble the material dried;

FIG. 5 is a vertical sectional view illustrating another drying apparatus in accordance with the invention in particularly showing a modified bed and skirt design;

FIG. 6 is an enlarged fragmentary sectional view depicting the interconnection between the bed and flexible skirt members in the FIG. 5 embodiment;

FIG. 7 is a vertical sectional view similar to that of FIG. 5, but showing another bed and skirt design wherein flexible skirts are stitched to the bed and at a location where an agitator is in its lowered position;

FIG. 8 is a vertical sectional view similar to that of FIG. 7 and showing the FIG. 7 design at a location where an agitator is in its uppermost position;

FIG. 9 is a fragmentary side view in vertical section illustrating another embodiment of an apparatus suited for use with the invention making use of an endless belt flexible bed;

FIG. 10 is a vertical sectional view taken along line 10-10 of FIG. 9 and depicting the FIG. 9 embodiment at a location where an agitator is in its lowered position; and

FIG. 11 is a vertical sectional view taken along the line 11-11 of FIG. 9 and depicting the FIG. 9 embodiment at a location where an agitator is in its upper position for elevating and tumbling material.

### DETAILED DESCRIPTION OF THE DRAWINGS

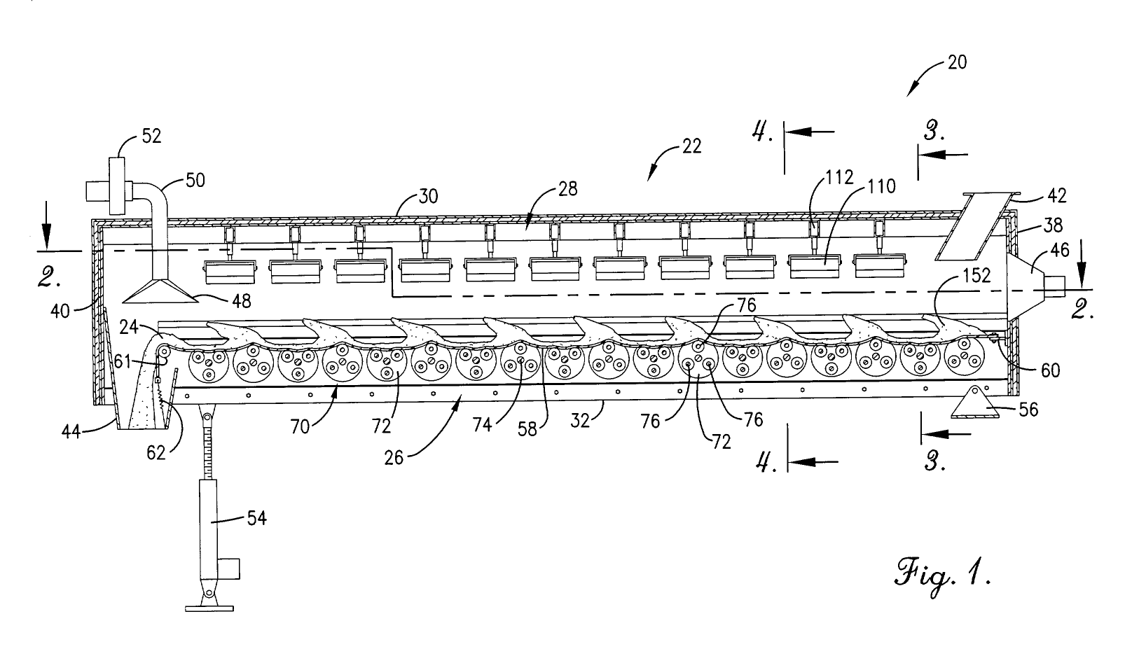

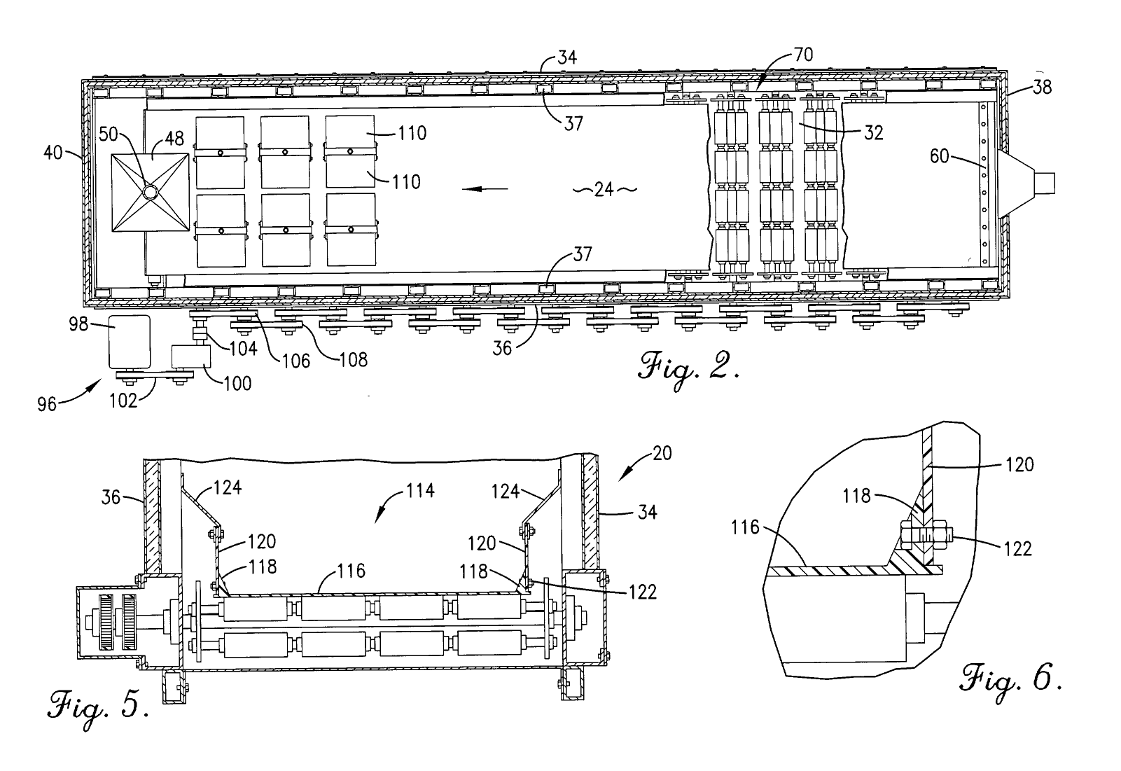

Turning now to the drawings, and particularly FIGS. 1-2, an agitated bed drying apparatus 20 suited for use in accordance with the invention is illustrated. Broadly speaking, the apparatus 20 includes an elongated housing 22, an essentially imperforate, flexible elongated bed 24 within the housing 22, an agitator assembly 26 beneath bed 24, and an infrared drying array 28 within the housing 22 and above the bed 24.

In more detail, the housing 22 includes elongated top, bottom and sidewalls 30, 32, 34 and 36, together with end walls 38 and 40. As shown, all of the walls 30-40 are insulated, save for bottom wall 32. In order to provide extra strength, the sidewalls 34, 36 and top wall 30 include axially spaced apart, box-type, interconnected frame members 37. The housing 22 is equipped with a material inlet 42 adjacent end wall 38, as well as a chute-type material outlet 44 proximal to the opposed end wall 40. An air handling system is provided with housing 22 and includes an inlet manifold 46 extending through end wall 38 above the bed 24, as well as an exhaust hood 48 adjacent outlet 44; the hood 48 is coupled with an exit pipe 50, the latter having an exhaust fan 52 interposed therein. A best seen in FIG. 1, a conventional jacking screw 54 is affixed to the outlet end of housing 22, while the inlet end thereof is pivotally supported via pivot mount 56. Thus, the inclination of the bed 24 can be selectively altered by manipulation of the jacking screw 54.

The bed 24 in the FIG. 1 embodiment is made up of an elongated, imperforate length of flexible material 58 which extends the full length of housing 22 as shown. The inlet end of the material 58 is fixedly secured to end wall 38 by means of fastener bar 60. The opposed outlet end of the material 58 is trained over and idler 61 and is yieldably attached to outlet 44 by means of a spring 62. In this fashion, the material 58 is maintained substantially in tension throughout the use of apparatus 20 and against any significant translational movement.

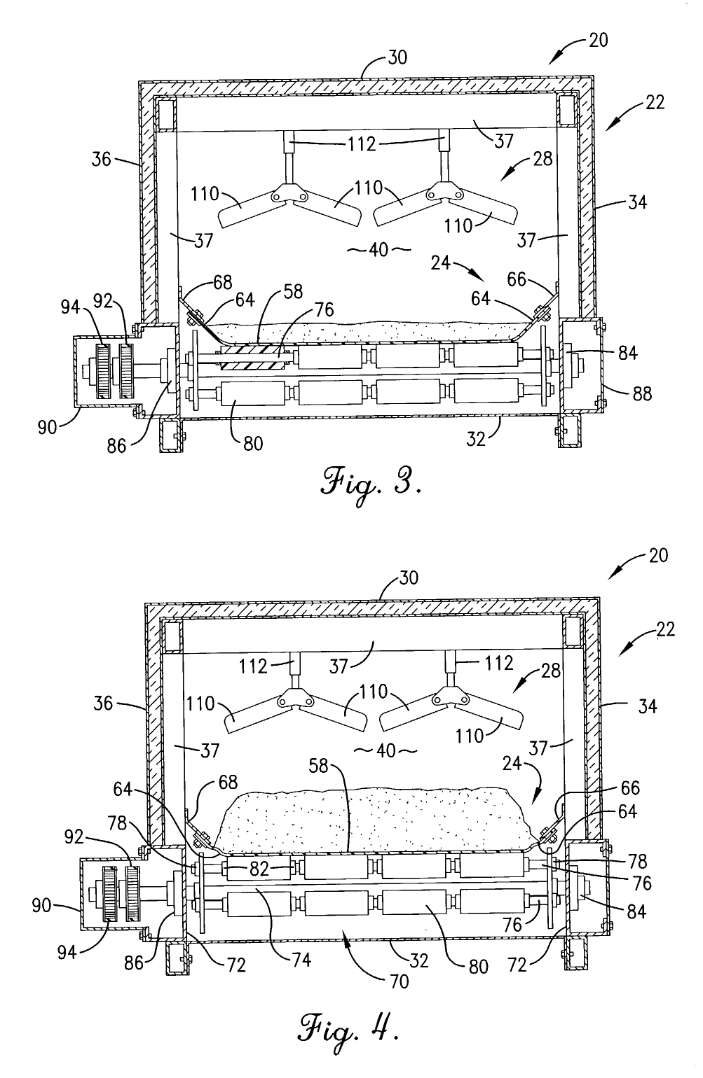

Referring to FIGS. 3 and 4, it will be seen that the side marginal edges 64 of the material 58 are secured to and supported by respective, elongated, obliquely oriented metallic connector elements 66 affixed to the upright frame members 37. Thus, the connector elements 68 and margins 64 cooperatively define upright skirt members associated with the bed 24 in order to retain material on the bed during drying operations.

The agitator assembly 26 is made Up of a series of elongated, transversely extending, axially spaced, multiple-bar rotatable beater bar units 70. The individual units 70 are identical and each includes a pair of circular end plates 72 together with an elongated central drive shaft 74 extending the full length of the unit 70 through the end plates 72. Each unit 70 further has a total of three roller bars 76 circumferentially and evenly spaced about the shaft 74 at 120° intervals. Each of the bars 76 is fixed to the plates 72 by set collars 78. As best viewed in FIGS. 3 and 4, each bar 76 supports a plurality, here four, of elongated, annular, rotatable synthetic resin rollers 80. These rollers 80 are coaxially mounted on the corresponding bars 76 and are rotatable relative thereto by means of roller end bearings 82.

All of the units 70 are driven for powered rotation during operation of apparatus 20. Again referring to FIGS. 3 and 4, it will be seen that the ends of drive shaft 74 are respectively received within bearings 84 and 86; the bearings 84 are located within an elongated bearing housing 88, whereas the bearings 86 are disposed within an opposed, elongated drive housing 90. The central drive shaft 74 of the unit 70 extends into the confines of drive housing 90 and support a pair of conventional drive belt pulleys 92 and 94.

A drive assembly 96 is provided for the agitator assembly 26 in order to effect rotation of the individual units 70. The assembly 96 has a variable speed motor 98 coupled to a reducer 100 via belt 102. The reducer is in turn connected by means of coupler 104 to the drive shaft 74 of the drive unit closest to outlet 44. This drive shaft is then connected to the adjacent drive shaft 74 through a drive belt 106. Alternating drive belts 108 respectively trained about adjacent pairs of drive pulleys 94 and 92 are employed for rotating all of the units 70, as will be readily apparent from a consideration of FIG. 2. Use of the variable speed motor 98 permits rotation of the beater bar unit 70 at different rotational speeds at the discretion of the user.

The units 70 are positioned relative to bed 24 so that, upon rotation thereof, each of the sets of rollers 80 of each unit successively impacts the underside of the flexible bed 24. This generates an undulating bed movement which serves to elevate and tumble the material to be dried on bed 24. This action is best illustrated in FIGS. 1, 3 and 4. In FIG. 3 the position of a unit 70 is illustrated wherein two of the rollers sets thereof are essentially parallel. In this orientation only a minimum amount of deflection of the bed 24 is occurring. In contrast (see FIG. 4) as the unit 70 is further rotated a roller set moves to its uppermost position, thereby more substantially deflecting the bed 24. This causes the material atop bed 24 to be elevated and tumbled in an arcuate and generally forward direction towards outlet 44. In essence, when each unit 70 moves between the FIG. 3 and FIG. 4 position, a vertical component of motion is imparted to the bed 24.

The infrared drying array 28 is made up of a number of infrared heaters 10 which are arranged in pairs, with each pair being supported by a mount 112 extending downwardly from top wall 30. The heater pairs extend essentially the full length of housing 22 between inlet 42 and hood 48. These heaters are themselves conventional, and are of a type described in U.S. Pat. No. 5,557,858, which is incorporated by reference herein. Generally speaking, the infrared heaters 110 are designed for emitting infrared radiation having a wavelength of from about 0.5-12 microns and more preferably from about 3-7 microns. The operating temperature range of these units is typically on the order of from about 300°-900° F.

FIGS. 5 and 6 illustrate a modified bed 114 for use in the apparatus 20. The bed 114 includes an elongated, resilient synthetic resin belt-like element 116 having side marginal upstands 118. A pair of elongated, fore and aft extending flexible cloth elements 120 are secured to the upstands 118 by means of bolt fasteners 122 (see FIG. 6). The upper ends of the cloth elements 122 are supported by elongated, metallic, obliquely oriented connectors 124 affixed to the housing sidewalls 34, 36. In this embodiment, the cloth elements 120 present skirt members for the bed 114. The connectors 124 assist in the skirting function, and also are designed for directing infrared energy from the heaters 110 to the material on bed 114.

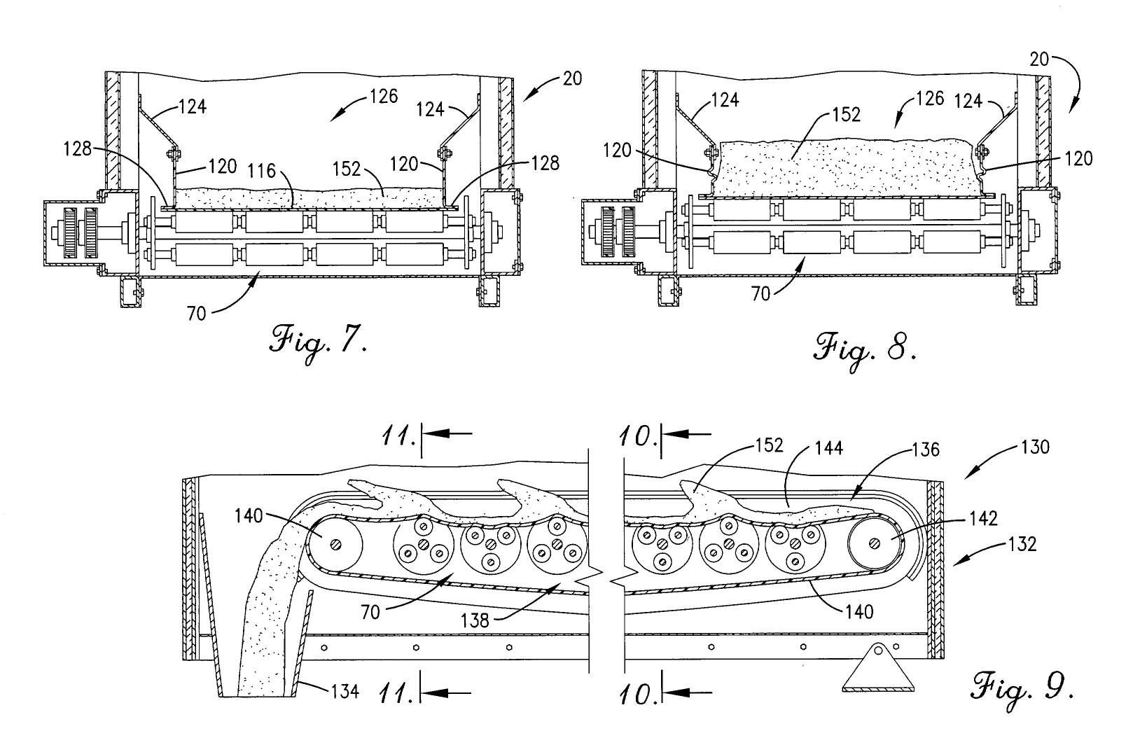

FIGS. 7 and 8 illustrate the use of yet another bed 126 in the apparatus 20. In this case the bed 126 is made up of the belt-like member 116 with upstanding cloth elements 120 affixed to the member 16 by longitudinal stitching lines 128. As in the previous embodiment, the upper ends of the elements 120 are supported by the sidewall mounted connectors 124. As shown in FIG. 8, when the individual beater bar units 70 are in their uppermost position for elevating and tumbling the material being dried, the cloth elements 120 fold as necessary to accommodate movement of the bed 126.

FIGS. 9-11 illustrate another drying apparatus 130 which in many respects is similar to apparatus 24. Thus, the overall apparatus 130 includes an insulated housing 132 having a material inlet (not shown) and a chute-type material outlet 134. A bed 136 is located within the housing 132, along with an agitator assembly 138 and an infrared drying array (not shown). In the case of apparatus 130, however, the bed 136 is in the form of an elongated, endless belt 140 which is trained around endmost rollers 140 and 142. Typically, the roller 140 is powered by a conventional drive so as to continuously move the belt 140.

The agitator assembly 138 is made up of a series of transversely extending, axially spaced apart beater bar units 70, as in the case of the FIG. 1 embodiment. Similarly, these units 70 are driven for rotation by the same type of drive assembly 96 previously described, and the apparatus 130 has an infrared drying array of the same character as the FIG. 1 embodiment.

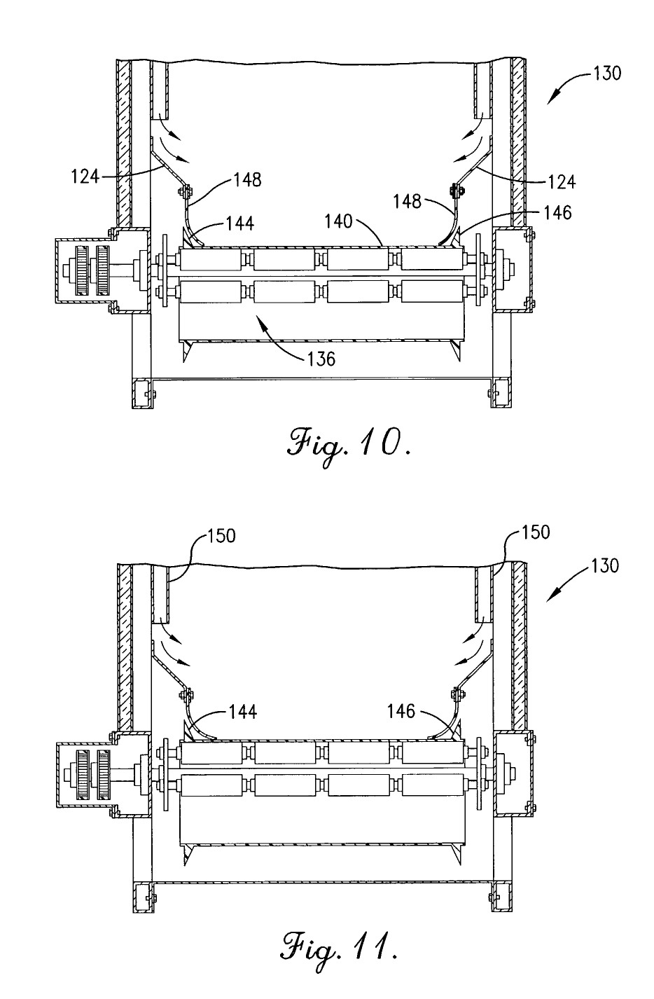

FIGS. 10 and 11 illustrate in more detail the bed 36 and particularly the skirting arrangement employed. In particular, the belt 140 includes a pair of outwardly projecting, continuous marginal cleats 144, 146. These cleats cooperate with a pair of upright cloth elements 148 which are located with their bottom ends inboard of the cleats 144, 146 and lying atop the upper run of the belt 140. As can be appreciated, the cloth elements 148 and cleats 144, 146 cooperatively define a material-retaining skirt member adjacent each marginal edge of the bed 136. The upper ends of the cloth elements 148 are supported on oblique connectors 124 previously described.

The apparatus 130 differs in one additional respect from the earlier embodiments. In this unit, a series of sidewall mounted, depending, open bottom, spaced apart air tubes 150 are mounted adjacent the inner surfaces of the housing sidewalls and are coupled to a source of pressurized air (not shown). Thus, during drying operations, pressurized air is directed downwardly and is diverted by the connectors 124 onto the material on the belt 140 for the purpose of enhancing the drying effect.

The source of pressurized air will largely depend on the desired temperature conditions within the housing 132. For example, it is possible to recirculate the hot air generated within the housing 132 by the infrared array 28, mix a quantity of ambient air with the air being recirculated from the housing 132, or even circulate entirely ambient air within the apparatus 20. It is preferable to incorporate a quantity of ambient air into the recirculating air mixture when the ambient air temperature is already warm. However, when the ambient air temperature is cooler, it is preferable to circulate only the hot air generated within the housing 132. One skilled in the art will readily be able to determine the precise quantity of ambient air, if any, to circulate within the housing 132.

The operation of all of the foregoing embodiments will be readily understood from the drawings and the preceding discussion. Generally speaking however, and referring to the FIG. 1 embodiment as an example, material 152 is fed through inlet 42 onto bed 24 during rotation of the agitator assembly 26 and while the array 28 is operating. The material 152 on the bed is successively moved along the length thereof through the action of the beater bar units 70. As each of the roller sets of the respective units 70 rotates to its maximum uppermost position, the bed 24 is deflected upwardly. This causes the material directly above on the bed 24 to be elevated and tumbled generally in a direction towards outlet 44. As illustrated in FIG. 1, this action of the agitator assembly 26 causes a “rolling” action serving to expose the material 152 to the radiation from the array 28. As the material 152 advances along the bed 24, it ultimately passes through chute outlet 44 for recovery as a dried product. During this time, positive pressure ambient air may be directed into the housing 22 through manifold 46, while moisture-laden air is exhausted through hood 48. In this fashion, the ambient atmosphere within housing 22 is maintained relatively dry to assist in processing of the material 152.

### EXAMPLES

The following examples describe preferred methods in accordance with the invention. It is to be understood that these examples are illustrations only and nothing therein should be deemed as a limitation upon the overall scope of the invention. An agitated bed drying apparatus constructed in accordance with the above description and U.S. Pat. No. 5,893,217 incorporated by reference herein was used in all of the following examples.

### Example 1

In this example, a series of tests according the method of the present invention were performed using freshly harvested rice. The rice used in the tests had initial moisture contents between about 15-18% by weight. In all tests, the temperature of the rice never exceeded 110° F. and averaged about 105° F. The results of the rice tests are set forth in Table 1.

TABLE 1

| | Test 1 | Test 2 | Test 3 | Test 4 |
|---|---|---|---|---|
| Starting Moisture | 15.2% | 18.1% | 17.4% | 17.9% |
| (by weight) | | | | |
| Ending Moisture | 12.8% | 12.4% | 12.6% | 11.3% |
| (by weight) | | | | |
| Starting Rice | 89° | 89° | 89° | 91° |
| Temperature | | | | |
| (° F.) | | | | |
| Ending Rice | 105° | 105° | 103° | 108° |
| Temperature | | | | |
| (° F.) | | | | |
| Starting Weight | 408 | 397 | 506 | 485 |
| (lbs.) | | | | |
| Ending Weight | 378 | 369 | 421 | 474 |
| (lbs.) | | | | |
| Milling Yield | (66/71.8) | (67.7/73.2) | (66.7/72.3) | (63.8/70.8) |
| (grams | 92% | 90% | 92% | 90% |
| unfractured | | | | |
| kernels/grams | | | | |
| total kernels) | | | | |

In tests 1 and 2, the rice was dried for a period of about 1.5-2.5 hours. In tests 3 and 4, the rice was dried for approximately 2.5-3.0 hours. At the end of that drying process, the rice had an average moisture content under 13% by weight. Moisture levels and rice temperatures taken every 30 minutes during the drying process for each test is shown in Table 2. All moisture levels were recorded on a Motomco Moisture Meter. This device is the same or similar to those that grain mills typically use in measuring rice moisture content. The rice milling yield was then determined.

TABLE 2

| Drying Time (minutes) | 0 | 30 | 60 | 90 | 120 | 150 | 180 |
|---|---|---|---|---|---|---|---|
| Test 1 | | | | | | | |
| Moisture Content (by weight) | 15.2% | 14.9% | 14.1% | 12.8% | — | — | — |
| Rice Temperature (° F.) | 89° | 108° | 105° | 104° | — | — | — |
| Test 2 | | | | | | | |
| Moisture Content (by weight) | 18.1% | 14.6% | 13.9% | 13.1% | 12.6% | 12.4% | — |
| Rice Temperature (° F.) | 89° | 103° | 103° | 105° | 103° | 105° | — |
| Test 3 | | | | | | | |
| Moisture Content (by weight) | 17.4% | 14.5% | 13.6% | 13.0% | 12.6% | 12.6% | — |
| Rice Temperature (° F.) | 89° | 97° | 97° | 100° | 100° | 103° | — |
| Test 4 | | | | | | | |
| Moisture Content (by weight) | 17.9% | 14.3% | 13.6% | 13.0% | 12.7% | 11.6% | 11.3% |
| Rice Temperature (° F.) | 91° | 102° | 104° | 104° | 105° | 105° | 108° |

To determine the milling yield, approximately 162 g samples of the rice from each test were milled to remove the hull and bran leaving just the rice kernel. The rice kernels were individually examined and the fractured kernels removed. The milling yield is the weight ratio of unfractured kernels/total kernels. The milling yield was 90% or above in each test. Stated another way, 10% or less of the rice kernels fractured during the drying process. Under normal industry standards, it is desirable to fracture less than about 22% of the rice kernels during the drying process. Therefore, the results of the rice drying tests according to the method of the present invention are considered excellent.

### Example 2

In this example, a series of tests were performed according to the method of the present invention using harvested rosemary, more specifically the tops of the rosemary plant. While the rosemary pieces used in this example were approximately 12-14 inches in length, it is possible to use shorter rosemary plant pieces, preferably 1-2 inches in length. The average moisture content of the rosemary prior to drying was about 58%. The rosemary was dried for about 2.5-3 hours. In all tests, the temperature of the rosemary never exceeded 130° F. The results of the rosemary drying tests are set forth in Table 3.

TABLE 3

| | Test 5 | Test 6 | Test 7 | Test 8 | Test 9 |
|---|---|---|---|---|---|
| Starting Moisture (by weight) | 64.2% | 57% | 53.2% | 55.6% | 58.2% |
| Ending Moisture (by weight) | 8.8% | 17% | 8.2% | 8% | 13.2% |
| Starting Rosemary Temperature (° F.) | 89° | 87° | 92° | 87° | 87° |
| Ending Rosemary Temperature (° F.) | 116° | 114° | 121° | 123° | 108° |
| Starting Weight (lbs.) | 119 | 149 | 174 | 119 | 119 |
| Ending Weight (lbs.) | 68 | 75 | N/A | 60 | 73 |

Tests 5, 7 and 8 were considered successful trials because the moisture content was reduced to less than 9% by weight. Tests 6 and 9 were not successful in that not enough moisture was removed from the rosemary. These unsatisfactory results were attributed to the adjustment of secondary variables such as the volume of air flow through and the exhausting of moisture from the drying apparatus.

### Example 3

This example involves the denaturation of the enzyme lipase in rice bran. Brown rice that has previously been dried is exposed to far-infrared radiation and heated to a temperature of about 200° F. for about 1-3 minutes. During this processing step, the lipase enzyme present in the rice bran is denatured and rendered inactive. The rice bran is then milled off of the rice kernel allowing the rice bran to be used for nutriceutical extraction and human consumption.

### Example 4

In this example, rice which has been previously dried to a moisture content of about 7-13% by weight is disinfested. The infested rice is exposed to far-infrared radiation and heated to a temperature of about 140° F. and maintained at that temperature for between about 1-3 minutes thereby killing the infesting insects and their larvae. The rice is then cooled to ambient temperature.

### Example 5

In this example, chicken feed is sterilized to kill salmonella bacteria. The chicken feed is exposed to far-infrared radiation and heated to a temperature of about 180° F. for about 1-3 minutes during which the salmonella bacteria present on the chicken feed is effectively destroyed. In so killing the bacteria, transmission of salmonella amongst the particular chicken population is prevented.

We claim:

1. A method of treating comestible material with infrared radiation comprising exposing the material to infrared radiation and heating the material up to a temperature of about 300° F., said exposing step comprising exposing substantially all of the outer surface of the material to said infrared radiation.

2. The method of claim 1, said exposing step being for sufficient time to disinfest the comestible material.

3. The method of claim 1, said exposing step being for sufficient time to denature an enzyme within the comestible material.

4. The method of claim 3, said enzyme being lipase or amylase.

5. The method of claim 1, said exposing step being for sufficient time to inactivate microorganisms and/or fungi on the comestible material.

6. The method of claim 1, said exposing step being for a period of less than about 3 minutes.

7. The method of claim 1, said exposing step comprising elevating and tumbling the material thereby exposing substantially all of the outer surface of the material to said infrared radiation.

8. The method of claim 1, heating the comestible material to a temperature between about 100°-200° F.

9. The method of claim 1, heating the comestible material to a temperature between about 140°-170° F.

10. The method of claim 1, said infrared radiation being far-infrared radiation.

11. The method of claim 1, said infrared radiation having a wavelength of about 2-7 microns.

12. A method of treating comestible material with infrared radiation comprising the steps of:
placing the material on an elongated, essentially imperforate flexible bed;
exposing the material to said infrared radiation and heating the material up to a temperature of about 300° F.; and
repeatedly impacting the lower surface of said bed in order to generate an undulating bed movement serving to elevate and tumble said material on said bed and to move the material along the length thereof.

13. The method of claim 12, said exposing step occurring for sufficient time to denature an enzyme present within the comestible material.

14. The method of claim 13, said enzyme being lipase or amylase.

15. The method of claim 12, said exposing step occurring for sufficient time to disinfest the comestible material.

16. The method of claim 15, said exposing step inactivating at least about 95% of all pests in the material.

17. The method of claim 12, said exposing step occurring for sufficient time to inactivate microorganisms and/or fungi on the comestible material.

18. The method of claim 17, said exposing step inactivating at least about 95% of all microorganisms and/or fungi on the comestible material.

19. The method of claim 12, said exposing step occurring for less than about 3 minutes.

20. The method of claim 19, said exposing step occurring for less than about 30 seconds.

21. The method of claim 12, heating the comestible material to a temperature of between 100°-200° F.

22. The method of claim 12, heating the comestible material to a temperature between about 140°-170° F.

23. The method of claim 12, said impacting step comprising the step of repeatedly contacting the lower surface of said bed with rotating elements.

24. The method of claim 12, said infrared radiation being far-infrared radiation.

25. The method of claim 12, said infrared radiation having a wavelength of about 2-7 microns.
